(19) United States
(12) Patent Application Publication
(10) Pub. No.: US 2011/0098795 A1
(43) Pub. Date: Apr. 28, 2011

(54) NEUROLOGIC SCREENING CONNECTOR

(75) Inventors: Bruce R. Mehdizadeh, Savage, MN (US); Farooq M. Francis, Somerset, WI (US); Daniel C. Oster, Blaine, MN (US)

(73) Assignee: Medtronic, Inc., Minneapolis, MN (US)

(21) Appl. No.: 12/906,606

(22) Filed: Oct. 18, 2010

Related U.S. Application Data
(60) Provisional application No. 61/255,573, filed on Oct. 28, 2009.

(51) Int. Cl. A61N 1/05

(52) U.S. Cl. 607/117

(57) ABSTRACT

A medical lead screening connector includes a housing, a plurality of electrical contacts disposed within the housing, and a lead receptacle element pivotly connected to the housing. The lead receptacle has a lead aperture along a length of the lead receptacle element. The lead receptacle element engages the plurality of electrical contacts in a first closed position and disengages from the plurality of electrical contacts in a second open position.

### RELATED APPLICATIONS

This application claims priority to U.S. Provisional Patent Application No. 61/255,573, filed on Oct. 28, 2009 and titled “NEUROLOGICAL SCREENING CONNECTOR”. The entire disclosure of U.S. Provisional Patent Application No. 61/255,573 is incorporated herein by reference.

### BACKGROUND

The medical device industry produces a wide variety of electronic and mechanical devices for treating patient medical conditions such as pacemakers, defibrillators, neurostimulators and therapeutic substance delivery pumps. Medical devices can be configured to be surgically implanted or connected externally to the patient receiving treatment. Clinicians use medical devices alone or in combination with therapeutic substance therapies and surgery to treat patient medical conditions. For some medical conditions, medical devices provide the best and sometimes the only therapy to restore an individual to a more healthful condition and a fuller life.

One type of medical device is an implantable neurological stimulation system that can be used to treat conditions such as pain, movement disorders, pelvic floor disorders, gastroparesis, and a wide variety of other medical conditions. The neurostimulation system typically includes a neurostimulator, a stimulation lead, and an extension. The neurostimulator can be connected to a stimulation lead that has one or more electrodes to deliver electrical stimulation to a specific location in the patient's body.

The lead and stylet combination are part of an implantable neurostimulation system. The neurostimulation lead is placed in the desired location of the body. The stylet wire and handle combination are used to give the lead stiffness during implantation and to aid in maneuvering the lead into the desired position. Once the lead is believed to be placed in the appropriate position within the body the lead, with or without the stylet, is coupled with a neurostimulation screening cable, which is connected to a neurostimulation screening device. The screening device can be programmed to send different combinations, strengths and frequencies of electrical stimulation to the patient. The screening cable provides a connection to, and electrical pathway between the stimulation lead or percutaneous extension and the neurostimulation screening device. The patient is questioned to determine if the stimulation covers the desired region of the body. If provided results are favorable the patient receives a temporary implant of the stimulation lead system. Either the stimulation lead or percutaneous extension is attached to the screening cable for a trial screening period so the patient can assess the efficacy of the system in normal life settings. The patient can be sent home with an external neurostimulator that sends electrical stimulation to the stimulation lead via the screening cable during the trial period. This trial period can range from 1 to 30 days depending on the physician and the country in which the trial occurs. This trial period is used to access the efficacy of the stimulation therapy for the patient.

### BRIEF SUMMARY

The present disclosure relates to a medical lead screening connector. In particular, the present disclosure relates to a medical lead screening cable that includes a lead receptacle element that is pivotly connected to a lead screening connector housing. The pivoting lead receptacle element allows a lead to be engaged or disengaged with contacts on the lead screening connector housing by moving the pivoting lead receptacle element closed position and open position.

In one illustrative embodiment, a medical lead screening connector includes a housing, a plurality of electrical contacts disposed within the housing, and a lead receptacle element pivotly connected to the housing. The lead receptacle has a lead aperture along a length of the lead receptacle element. The lead receptacle element engages the plurality of electrical contacts in a first closed position and disengages from the plurality of electrical contacts in a second open position.

These and various other features and advantages will be apparent from a reading of the following detailed description.

### BRIEF DESCRIPTION OF THE DRAWINGS

The disclosure may be more completely understood in consideration of the following detailed description of various embodiments of the disclosure in connection with the accompanying drawings, in which:

FIG. 1 is a schematic diagram of a neurological lead implanted within a human body or patient;

FIG. 2 is a schematic diagram of an illustrative screening cable;

FIG. 3 is a schematic diagram of an illustrative screening cable housing with the cover removed; and

FIG. 4 is a schematic diagram of an illustrative screening cable housing with the cover opened.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

In the following description, reference is made to the accompanying set of drawings that form a part hereof and in which are shown by way of illustration several specific embodiments. It is to be understood that other embodiments are contemplated and may be made without departing from the scope or spirit of the present disclosure. The following detailed description, therefore, is not to be taken in a limiting sense.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “lower”, “upper”, “beneath”, “below”, “above”, and “on top”, if used herein, are utilized for ease of description to describe spatial relationships of an element(s) to another. Such spatially related terms encompass different orientations of the device in use or operation in addition to the particular orientations depicted in the figures and described herein. For example, if a cell depicted in the figures is turned over or flipped over, portions previously described as below or beneath other elements would then be above those other elements.

As used herein, when an element, component or layer for example is described as being “on” “connected to”, “coupled with” or “in contact with” another element, component or layer, it can be directly on, directly connected to, directly coupled with, in direct contact with, or intervening elements, components or layers may be on, connected, coupled or in contact with the particular element, component or layer, for example. When an element, component or layer for example is referred to as begin “directly on”, “directly connected to”, “directly coupled with”, or “directly in contact with” another element, there are no intervening elements, components or layers for example.

The present disclosure relates to a medical lead screening connector. In particular, the present disclosure relates to a medical lead screening cable that includes a lead receptacle element that is pivotly connected to a lead screening connector housing. The pivoting lead receptacle element allows a lead to be engaged or disengaged with contacts on the lead screening connector housing by moving the pivoting lead receptacle element closed position and open position. While the present disclosure is not so limited, an appreciation of various aspects of the disclosure will be gained through a discussion of the examples provided below.

The teachings presented herein are applicable to any implantable medical device system employing lead for delivering electrical signals to a tissue of a patient. For example, the system may include a neurostimulator, such as a peripheral nerve stimulator, a spinal cord stimulator, or a deep brain stimulator; a cardiac pacemaker or defibrillator; a gastric stimulator; or the like. It will be understood that the systems and devices described herein may be readily applied to systems employing leads for purposes of screening, sensing, monitoring, recording, or the like.

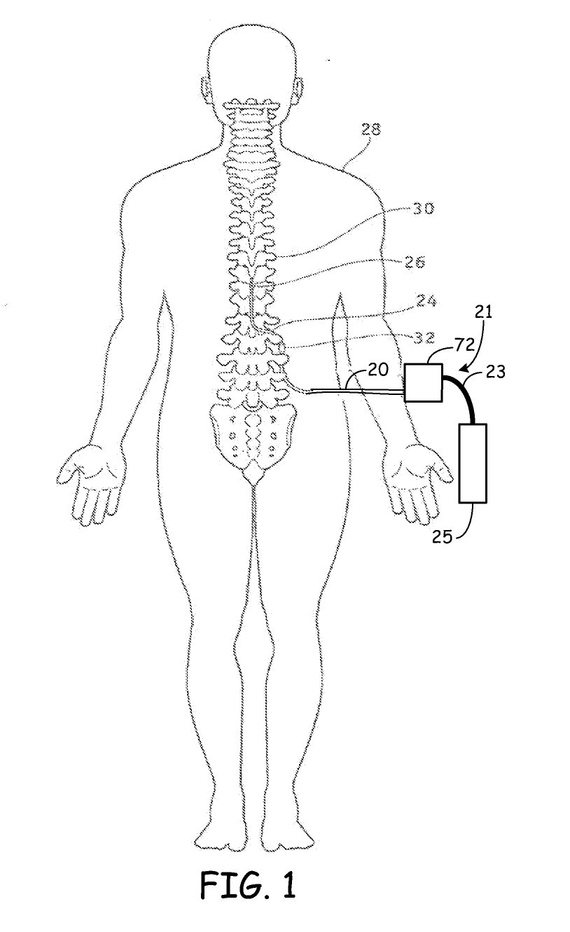

FIG. 1 is a schematic diagram of a neurological lead 24 implanted within a human body or patient 28. The implanted neurological lead 24 is a medical wire with special insulation. The neurological lead 24 includes one or more insulated electrical conductors with a connector on the proximal end and electrical contacts on the distal end. Some neurological leads are designed to be inserted into a patient percutaneously, and some neurological leads are designed to be surgically implanted. The neurological lead 24 may also be a paddle having a plurality of electrodes. Those skilled in the art will appreciate that any variety of neurological leads 24 may be used.

The neurological lead 24 can be implanted and positioned to stimulate a specific site in the spinal cord 30 or the nervous system. The neurological lead 24 includes one or more electrodes 26 (small electrical contacts) through which electrical stimulation is delivered from either an external stimulator 25 or an implanted stimulator (not shown) to the targeted neural tissue. The external stimulator 25 or an implanted stimulator can be any “active medical device” or “signal generator” as described above and can be placed external to or in any location within a body cavity or tissue within the body, or on the surface of a patient's skin, as desired.

The external stimulator 25 can be used to test the efficacy of stimulation therapy for the patient before an implantable stimulator is surgically implanted. The external stimulator 25 is used in conjunction with a screening cable 21 which accepts the stimulation lead 24 or lead extension 20 and creates an electrical pathway to the external stimulator 25. The screening cable 21 includes a conductor cable 23 and a distal screening connector 72 that includes pivoting lead receptacles (described below). The conductor cable 23 electrically connects the lead receptacles within the distal screening connector 72 to the external stimulator 25.

The illustrated external stimulator 25 is coupled to a lead extension 20 via the screening cable 21. The lead extension 20 has a proximal end coupled to the screening cable 21, and a lead 24 having a proximal end coupled to a distal end 32 of the lead extension 20 and a distal end of the lead 24 coupled to one or more electrodes 26. In some embodiments, the lead 24 proximal end is coupled to the screening cable 21, without a need for a lead extension. In many embodiments, the screening cable 21 couples to one or two or more leads each having four to eight electrodes. Such a system may also include a physician programmer and a patient programmer (not shown). The external stimulator 25 can be considered to be a signal generator of the type available from Medtronic, Inc. and capable of generating multiple signals occurring either simultaneously or one signal shifting in time with respect to the other, and having independently varying amplitudes and signal widths. The external stimulator 25 can contain a power source and the electronics for sending precise, electrical signals to the patient to provide the desired treatment therapy. While the external stimulator 25, in many embodiments, provides electrical stimulation by way of signals, other forms of stimulation may be used as continuous electrical stimulation.

In many embodiments, the lead 24 is a wire having insulation thereon and includes one or more insulated electrical conductors each coupled at their proximal end to a connector and to contacts/electrodes 26 at its distal end. Some leads are designed to be inserted into a patient percutaneously, and some are designed to be surgically implanted. In some embodiments, the lead 24 may contain a paddle at its distant end for housing electrodes 26. In many embodiments, electrodes 26 may include one or more ring contacts at the distal end of lead 24.

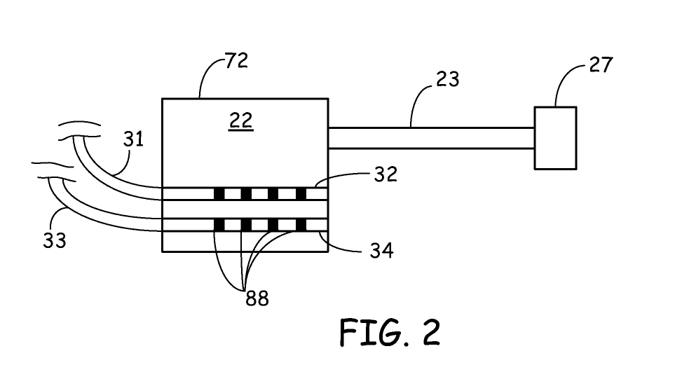

FIG. 2 is a schematic diagram of an illustrative screening cable 21. The screening cable 21 includes a proximal screening connector 27 electrically coupled to lead receptacles 32, 34 in the lead connector housing 22 of the distal screening connector 72. Conductor wires are disposed within the conductor cable 23 electrically coupled to the lead receptacles 32, 34 to the proximal screening connector 27. While two lead receptacles are illustrated, it is understood that the distal screening connector 72 can include any number of lead receptacles, as desired. In some embodiments, the distal screening connector 72 can be directly electrically coupled to a lead screening device or programming device (e.g., external stimulation device).

A first implantable medical lead 31 and second implantable medical lead 33 are received in the lead receptacles 32, 34. The lead receptors 32, 34 are configured for receiving the first implantable medical lead 31 and a second implantable medical lead 33. Electrical contacts on the lead 31, 33 mates with electrical contacts 88 within the distal screening connector 72.

The first implantable medical lead 31 and a second implantable medical lead 33 can be a wide variety of medical leads, such as a neurological lead. In some embodiments the medical lead can be a four-conductor neurological lead, a four-conductor extension, a four-conductor neurological lead with stylet handle, a four conductor percutaneous extension with stylet handle, an eight-conductor neurological lead, an eight-conductor extension, an eight-conductor neurological lead with stylet handle, and the like.

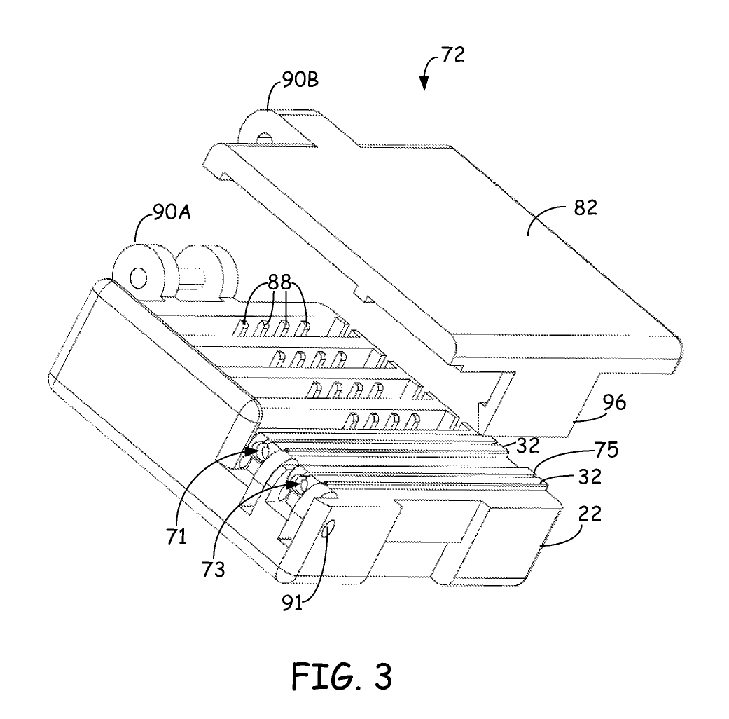

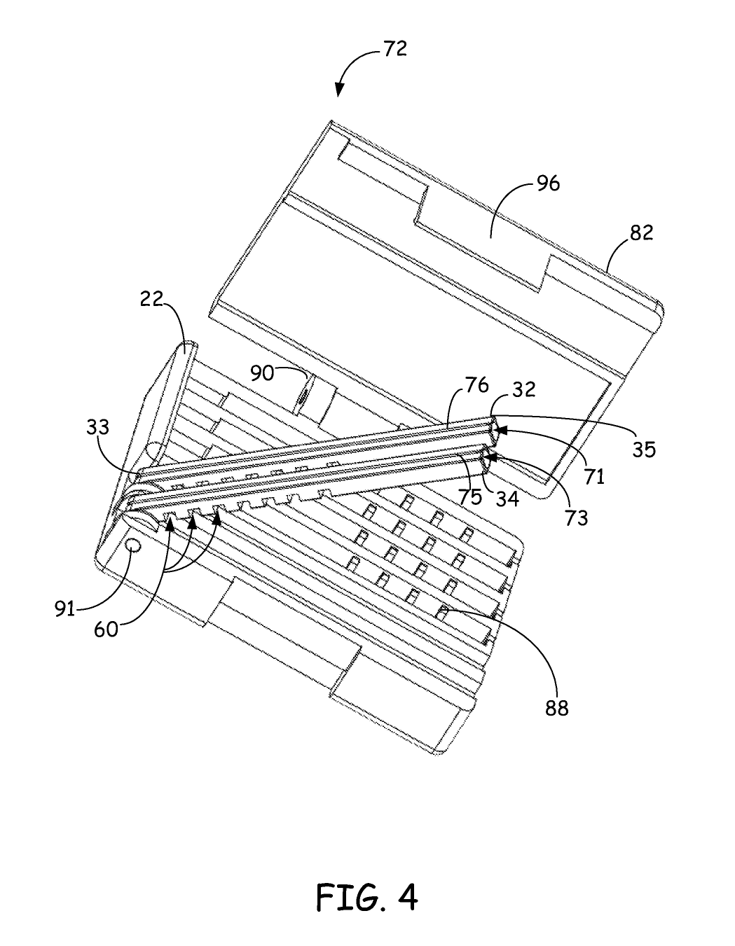

FIG. 3 is a schematic diagram of an illustrative screening connector 72 with the cover 82 removed. FIG. 4 is a schematic diagram of an illustrative screening connector 72 with the cover 82 opened. While the screening connector 72 is shown having two pivoting lead receptacle elements 32, 34, it is understood that the screening connector 72 can have any useful number of pivoting lead receptacle elements 32, 34.

The medical lead screening connector 72 includes a housing 22, a plurality of electrical contacts 88 disposed within the housing 22, and a lead receptacle element 32, 34 pivotly connected to the housing 22. The lead receptacle 32, 34 has a lead aperture 71, 73 along a length of the lead receptacle element 32, 34. The lead receptacle element 32, 34 engages the plurality of electrical contacts (not shown) in a first closed position and disengages from the plurality of electrical contacts in a second open position.

The lead receptacle element 32 is pivotly attached to the housing 22 at either a first end 33 or a second end 35 of the lead receptacle element 32. The lead receptacle element 32 pivots between a first closed position (see FIG. 3) and an open position (see FIG. 4) about a pivot point 91. In many embodiments, an implantable medical lead can be loaded into the lead receptacle element in the open position (often with a stylet loaded in the lead) and then the lead receptacle element is pivoted to the closed position to engage the contacts on the lead with the contacts on the medical lead screening connector 72.

In many embodiments the lead receptacle element 32, 34 includes a plurality of electrical contact apertures 60 along the length of the lead receptacle element 32, 34 and the plurality of electrical contact apertures 60 engage the plurality of electrical contacts (not shown) on the medical lead screening connector 72 in the closed position. In some embodiments there are four or eight electrical contact apertures 60 along the length of the lead receptacle element 32, 34. In many embodiments the electrical contact apertures 60 mate with the plurality of electrical contacts (not shown) on the medical lead screening connector 72 in the closed position. In many embodiments the lead receptacle element 32, 34 mechanically secures to the housing 22 of the medical lead screening connector 72 in the closed position via, for example, a friction fit or a latch element (not shown).

The housing 22 can include a cover 82 hingedly attached to the housing 22 and the cover 82 secures the lead receptacle element 32, 34 in the closed position. The cover 82 is coupled to the housing 22 via a hinge 90. The hinge 90 can include a housing hinge portion 90A and a cover hinge portion 90B. The cover 82 has a cover open position for permitting access to the lead receptacle element 32, 34 and a cover closed position for enclosing the lead receptacle element 32, 34 and securing the lead receptacle element 32, 34 in the closed position. The cover 82 can include a closing latch 96 that secures the cover 82 to the housing 22. In many embodiments the cover 82 is constructed from polymer materials and materials that are transparent to visible light.

In some embodiments, the lead receptacle 32, 34 are spring loaded such that when the cover 82 is opened, a resilient or spring member assists in pivoting the lead receptacle element 32, 34 away from the housing 22.

In many embodiments, the lead receptacle element 32, 34 includes a slit 76, 75 along the length of the lead receptacle element 32, 34. The slit can allow a stylet loaded onto a lead to pass along the length of the lead receptacle element 32, 34 and to assist in the loading of the lead into the pivoting lead receptacle element 32, 34.

Thus, embodiments of the NEUROLOGICAL SCREENING CONNECTOR are disclosed. The implementations described above and other implementations are within the scope of the following claims. One skilled in the art will appreciate that the present disclosure can be practiced with embodiments other than those disclosed. The disclosed embodiments are presented for purposes of illustration and not limitation, and the present invention is limited only by the claims that follow.

What is claimed is:

1. A medical lead screening connector comprising:
a housing;
a plurality of electrical contacts disposed within the housing; and
a lead receptacle element pivotly connected to the housing, the lead receptacle having a lead aperture along a length of the lead receptacle element, wherein the lead receptacle element engages the plurality of electrical contacts in a first closed position and disengages from the plurality of electrical contacts in a second open position.

2. A medical lead screening connector according to claim 1, wherein the lead receptacle element includes a plurality of electrical contact apertures along the length of the lead receptacle element and the plurality of electrical contact apertures engage the plurality of electrical contacts in the closed position.

3. A medical lead screening connector according to claim 1, wherein the housing further comprises a cover hingedly attached to the housing and the cover secures the lead receptacle element in the closed position.

4. A medical lead screening connector according to claim 3, wherein the cover is transparent to visible light.

5. A medical lead screening connector according to claims 1, further comprising a second lead receptacle element pivotly connected to the housing.

6. A medical lead screening connector according to claims 1, wherein the lead receptacle element includes a slit along the length of the lead receptacle element.

7. A medical lead screening connector according to claims 1, wherein the lead receptacle lead aperture is configured to accept an implantable lead and electrically connect the implantable lead with the plurality of electrical contacts disposed within the housing when the lead receptacle element is in the first closed position.

8. A medical lead screening connector according to claim 2, wherein the lead receptacle lead aperture is configured to accept an implantable lead and electrically connect the implantable lead with the plurality of electrical contacts disposed within the housing via the plurality of electrical contact apertures when the lead receptacle element is in the first closed position.

9. A medical lead screening connector according to claims 1, wherein the lead receptacle element secures to the housing in the closed position.

10. A medical lead screening connector according to claims 1, wherein the lead receptacle element length extends between a first end and a second end and the lead receptacle element is pivotly connected to the housing at the first end or the second end of the lead receptacle element.

11. A medical lead screening connector according to claim 7, wherein the implantable lead secures to the lead receptacle element in the closed position.

12. A medical lead screening cable comprising:
a proximal screening connector;
at least two screening conductors electrically connected to the proximal screening connector; and
a distal lead screening connector configured to receive at least two implantable medical leads, the distal lead screening connector electrically connected to the screening conductors and comprising:
a housing;
a plurality of electrical contacts disposed within the housing; and
a lead receptacle element pivotly connected to the housing, the lead receptacle having a lead aperture along a length of the lead receptacle element, wherein the lead receptacle element engages the plurality of electrical contacts in a first closed position and disengages from the plurality of electrical contacts in a second open position.

13. A medical lead screening cable according to claim 12, wherein the lead receptacle element includes a plurality of electrical contact apertures along the length of the lead receptacle element and the plurality of electrical contact apertures engage the plurality of electrical contacts in the closed position.

14. A medical lead screening cable according to claims 12, wherein the housing further comprises a cover hingedly attached to the housing and the cover secures the lead receptacle element in the closed position.

15. A medical lead screening cable according to claim 14, wherein the cover is transparent to visible light.

16. A medical lead screening cable according to claims 12, further comprising a second lead receptacle element pivotly connected to the housing.

17. A medical lead screening cable according to claims 12, wherein the lead receptacle element includes a slit along the length of the lead receptacle element.

18. A medical lead screening cable according to claims 12, wherein the lead receptacle lead aperture is configured to accept an implantable lead and electrically connect the implantable lead with the plurality of electrical contacts disposed within the housing when the lead receptacle element is in the first closed position.

19. A medical lead screening cable according to claim 13, wherein the lead receptacle lead aperture is configured to accept an implantable lead and electrically connect the implantable lead with the plurality of electrical contacts disposed within the housing via the plurality of electrical contact apertures when the lead receptacle element is in the first closed position.

20. A medical lead screening cable according to claims 12, wherein the lead receptacle element secures to the housing in the closed position.

21. A medical lead screening cable according to claims 12, wherein the lead receptacle element length extends between a first end and a second end and the lead receptacle element is pivotly connected to the housing at the first end or the second end of the lead receptacle element.

22. A medical lead screening cable according to claim 18, wherein the implantable lead secures to the lead receptacle element in the closed position.
